(19) United States
(12) Patent Application Publication
(10) Pub. No.: US 2015/0216850 A1
(43) Pub. Date: Aug. 6, 2015

(54) COMBINATION OF RASAGILINE AND PRIDOPIDINE FOR TREATING NEURODEGENERATIVE DISORDERS, IN PARTICULAR HUNTINGTON'S DISEASE

(71) Applicants: Michael HAYDEN, Petach Tikva (IL); Cheryl FITZER-ATTAS, Rehovot (IL)

(72) Inventors: Michael Hayden, Petach-Tikva (IL); Cheryl Fitzer-Attas, Rehovot (IL)

(21) Appl. No.: 14/426,339

(22) PCT Filed: Sep. 27, 2013
(86) PCT No.: PCT/US2013/062484

§ 371 Date: Mar. 5, 2015

Related U.S. Application Data
(60) Provisional application No. 61/879,007, filed on Sep. 17, 2013.
(60) Provisional application No. 61/706,711, filed on Sep. 27, 2012.

(51) Int. Cl. A61K 31/451; A61K 31/135

(52) U.S. Cl. CPC A61K 31/451; A61K 31/135

(57) ABSTRACT

This invention provides a method of treating a patient afflicted with a neurodegenerative disorder, e.g., Huntington's disease, comprising administering to the patient rasagiline as an add-on therapy to or in combination with pridopidine. This invention also provides a package and a pharmaceutical composition comprising rasagiline and pridopidine for treating a patient afflicted with a neurodegenerative disorder. This invention also provides rasagiline for use as an add-on therapy or in combination with pridopidine in treating a patient afflicted with a neurodegenerative disorder. This invention further provides use of rasagiline and pridopidine in the preparation of a combination for treating a patient afflicted with a neurodegenerative disorder.

This application claims priority of U.S. Provisional Application No. 61/879,007 filed Sep. 17, 2013, and U.S. Provisional Application No. 61/706,711, filed Sep. 27, 2012, the entire contents of which are hereby incorporated by reference herein.

Throughout this application, various publications are referred to by first author and year of publication. Full citations for these publications are presented in a References section immediately before the claims. Disclosures of the documents and publications cited are hereby incorporated by reference in their entireties into this application in order to more fully describe the state of the art as of the date of the invention described herein.

### BACKGROUND

### Huntington's Disease (HD)

Huntington's disease (HD) is an inherited disease of the central nervous system that is characterized by chorea and progressive cognitive deterioration. Symptoms and signs of HD develop insidiously, starting at about age 35-50 but can develop before adulthood. Dementia or psychiatric disturbances (e.g., depression, apathy, irritability, anhedonia, antisocial behavior, full-blown bipolar or schizophreniform disorder) develop before or simultaneously with the movement disorder. Symptoms also include abnormal movements, such as myoclonic jerks or irregular movements of extremities, a lilting gait, facial grimacing, ataxia and inability to sustain motor act (motor impersistence) such as tongue protrusion. As the disease progresses, walking and swallowing become more difficult and the dementia becomes more severe. Most HD patients will eventually require institutionalization, and death usually occurs 13-15 years after the symptoms begin, usually due to an intercurrent infection (Tyagi et al., 2010; The Merck Manual).

HD is an autosomal dominant disorder resulting from a gene mutation causing abnormal repetition of the DNA sequence CAG which codes for the amino acid glutamine. The resulting huntingtin protein is a mutant huntingtin (mHtt) with an expanded stretch of polyglutamine residues, leading to the disease via unknown mechanism (The Merck Manual).

There is currently no cure for HD. In addition, tetrabenazine is the only medication currently approved by the Food and Drug Administration (FDA) to treat the symptoms of Huntington's disease. However supportive therapies are currently available to manage the symptoms.

Symptomatic treatment of Huntington's disease involves use of dopamine antagonists, presynaptic dopamine depleters, antidepressants, tranquilizers, anxiolytic benzodiazepines, anticonvulsants and antibiotics. Chorea and agitation may be partially suppressed by antipsychotics (e.g., chloropromazine 25-300 mg pot id, haloperidol 5-45 mg po bid); dose is increased until intolerable or undesirable adverse effects (e.g., lethargy, parkinsonism) occur. Alternatively, tetrabenazine may be used. The dose starts at 12.5 mg po once/day, and is subsequently increased (to 12.5 mg bid in the second week, 12.5 tid in the third week, up to a total of 100 mg/day divided into 3 doses) until intolerable adverse effects (e.g., sedation, akathisias, parkinsonism, depression) occur or chorea resolves (Tyagi et al., 2010; The Merck Manual).

Several medications including baclofen, idebenone and vitamin E have been studied in clinical trials with limited samples. Some experimental therapies for HD have aimed to reduce glutamatergic neurotransmission via the N-methyl-D-aspartate receptor and bolster mitochondrial energy production. However, currently no drug has been recommended for HD (Tyagi et al., 2010; The Merck Manual).

### Rasagiline

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514, 7,750,051, and 7,855,233 disclose R(+)-N-propargyl-1-aminoindan (“R-PAI”), also known as rasagiline, and its pharmaceutically acceptable salts. These U.S. patents also disclose that rasagiline is a selective inhibitor of the B-form of the enzyme monoamine oxidase (“MAO-B”) and is useful in treating Parkinson's disease and various other conditions by inhibition of MAO-B in the brain.

U.S. Pat. Nos. 6,126,968, 7,572,834, and 7,598,420, U.S. patent application Ser. Nos. 12/283,022, and 12/283,107 and PCT publications WO 95/11016 and WO 2006/014973, hereby incorporated by reference, disclose pharmaceutical compositions comprising rasagiline and processes for their preparation.

AZILECT® is a commercially available rasagiline mesylate immediate release formulation indicated for the treatment of the signs and symptoms of idiopathic Parkinson's disease as initial monotherapy and as adjunct therapy to levodopa. The current marketed formulation of rasagiline (Azilect®) is rapidly absorbed, reaching peak plasma concentration (tₘₐₓ) in approximately 1 hour. The absolute bioavailability of rasagiline is about 36%. (AZILECT® Product Label, May 2006).

### Pridopidine

Pridopidine (ACR16, Huntexil®, 4-[3-(methylsulfonyl)phenyl]-1-propyl-piperidine]) is a dopamine receptor mixed antagonist/agonist (US 2011/0206782). Pridopidine shows benefits in treating neurodegenerative disorders including Huntington's disease (Miller & Bezprozvanny 2010).

Pridopidine acts on central dopamine D2 receptors to potentially improve voluntary motor function in Huntington's disease patients (Venuto, 2012). The method of action is still not precisely known but pridopidine may stimulate or inhibit dopamine to normalize hypo- and hyper-dopaminergic behavior (Miller & Bezprozvanny 2010).

Huntexil® is the brand name for pridopidine developed by Neurosearch, Denmark to treat movement and psychiatric disorders (Miller & Bezprozvanny 2010). A recent MermaiHD Phase III clinical trial in Europe showed benefits from a treatment of 45 mg daily, or 90 mg daily (45 mg administered twice daily) for 6 months in Huntington's disease patients. Amounts of pridopidine up to 90 mg per day were well tolerated in Huntington's disease patients. The primary endpoint was the effect of Huntexil® on a specific subset of motor symptoms defined in the mMS at 26 weeks and was not met. However, the tertiary endpoint, UHDRS-TMS measuring changes in motor function, and individual items within the mMS (including gait and dysarthria) found a statistically significant effect of treatment (de Yebenes, 2011). Huntexil® slowed Huntington's disease symptoms and may have slowed Huntington's disease progression (Miller & Bezprozvanny 2010). The HART trial, initial Phase IIb studies in the United States and Canada, showed a significant effect on total motor function after twice-daily doses of 45 mg over 12 weeks (NeuroSearch—The HART Study). Clinical trials in the United States are ongoing to assess the long-term safety and treatment effects (Clinical Trials: OPEN-HART, 2011).

### Add-on/Combination Therapy

The effects of add-on or combination therapy using rasagiline and pridopidine on Huntington's disease patients have not been reported.

The administration of two drugs to treat a given condition, such as Huntington's disease, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of pridopidine and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of an add-on or combination therapy of two drugs, in particular rasagiline and pridopidine, cannot be predicted until the results of a formal combination study are available.

### SUMMARY OF THE INVENTION

This invention provides for a method of treating a human patient afflicted with neurodegenerative disorder comprising periodically administering to the patient an amount of rasagiline and an amount of pridopidine, wherein the amounts when taken together are effective to treat the human patient.

This invention also provides a package comprising a) a first pharmaceutical composition comprising an amount of rasagiline and a pharmaceutically acceptable carrier; b) a second pharmaceutical composition comprising an amount of pridopidine and a pharmaceutically acceptable carrier; and c) instructions for use of the first and second pharmaceutical compositions together to treat a human patient afflicted with a neurodegenerative disorder.

The invention also provides rasagiline for use as an add-on therapy or in combination with pridopidine in treating a human patient afflicted with Huntington's disease.

The invention also provides a pharmaceutical composition comprising an amount of rasagiline and an amount of pridopidine for use in treating a human patient afflicted with a neurodegenerative disease, wherein the rasagiline and the pridopidine are administered simultaneously or contemporaneously.

The invention also provides a pharmaceutical composition comprising an amount of rasagiline and an amount of pridopidine.

The invention also provides a use of an amount of rasagiline and an amount of pridopidine in the preparation of a combination for treating a human patient afflicted with a neurodegenerative disorder wherein the rasagiline or pharmaceutically acceptable salt thereof and the pridopidine are administered simultaneously or contemporaneously.

The invention also provides a pharmaceutical composition comprising an amount of rasagiline for use in treating a subject afflicted with a neurodegenerative disorder as an add-on therapy or in combination with pridopidine by periodically administering the pharmaceutical composition and the pridopidine to the subject.

The invention also provides a pharmaceutical composition comprising an amount of pridopidine for use treating a subject afflicted with a neurodegenerative disorder as an add-on therapy or in combination with rasagiline by periodically administering the pharmaceutical composition and the rasagiline to the subject.

### DETAILED DESCRIPTION OF THE INVENTION

This invention provides for a method of treating a human patient afflicted with neurodegenerative disorder comprising periodically administering to the patient an amount of rasagiline and an amount of pridopidine, wherein the amounts when taken together are effective to treat the human patient.

In one embodiment, the amount of rasagiline and the amount of pridopidine when taken together is more effective to treat the human patient than when each agent is administered alone.

In one embodiment, each of the amount of rasagiline when taken alone, and the amount of pridopidine when taken alone, is effective to treat the human patient. In another embodiment, either the amount of rasagiline when taken alone, or the amount of pridopidine when taken alone is not effective to treat a human patient.

In one embodiment, the neurodegenerative disorder is a trinucleotide repeat disorder. In another embodiment, the neurodegenerative disorder is a polyglutamine disease. In another embodiment the neurodegenerative disorder is a proteinopathy. In another embodiment the neurodegenerative disorder is Parkinson's disease, Alzheimer's disease, Amyotorphic lateral sclerosis (ALS) or Huntington's disease.

In one embodiment, the neurodegenerative disorder is Huntington's disease.

In one embodiment, the amount of rasagiline and the amount of pridopidine when taken together is effective to reduce a symptom of the neurodegenerative disorder in the human patient. In another embodiment, the symptom is depression, anxiety, motor function impairment, cognitive impairment, a physical symptom, a mental symptom, an emotional symptom, a behavioral symptom, impairment of the patient's functional capacity or reduced lifespan. In another embodiment, the symptom is motor function impairment. In another embodiment, the motor function impairment is abnormal movements, myoclonic jerks, irregular movements of extremities, gait, facial grimacing, ataxia, inability to sustain motor act, hand movement or balance.

In one embodiment, the patient's motor function is assessed by the Unified Huntington's Disease Rating Scale (UHDRS, TMS) or the modified motor score (mMS) derived from the Unified Huntington's Disease Rating Scale (UHDRS, TMS). In another embodiment the patient had an mMS score of 10 or greater at baseline.

In one embodiment, rasagiline is rasagiline mesylate.

In one embodiment, the administration of rasagiline substantially precedes the administration of pridopidine. In another embodiment, the administration of pridopidine substantially precedes the administration of rasagiline.

In one embodiment, the administration of rasagiline is 0 minutes to 48 hours after the administration of pridopidine. In another embodiment, the administration of rasagiline is 3-5 hours after the administration of pridopidine. In another embodiment, the administration of pridopidine is 0 minutes to 48 hours after the administration of rasagiline. In another embodiment, the administration of pridopidine is 3-5 hours after the administration of rasagiline.

In one embodiment, the human patient is receiving pridopidine therapy prior to initiating rasagiline therapy. In another embodiment, the human patient is receiving rasagiline therapy prior to initiating pridopidine therapy.

In one embodiment, the administration of rasagiline and pridopidine improves a symptom of a neurodegenerative disorder by at least 30%. In another embodiment, the administration of rasagiline and pridopidine improves a symptom of a neurodegenerative disorder by at least 50%. In another embodiment, the administration of rasagiline and pridopidine improves a symptom of a neurodegenerative disorder by more than 100%. In another embodiment, the administration of rasagiline and pridopidine improves a symptom of a neurodegenerative disorder by more than 300%.

In another embodiment, the administration of rasagiline and pridopidine improves a symptom of a neurodegenerative disorder by more than 1000%.

In one embodiment, the rasagiline is administered via oral administration.

In one embodiment, the rasagiline is administered daily. In another embodiment, the rasagiline is administered more often than once daily. In another embodiment, the rasagiline is administered less often than once daily.

In one embodiment, the amount of rasagiline administered is less than 0.5 mg/day. In another embodiment, the amount of rasagiline administered is 0.01-20.0 mg/day. In another embodiment, the amount of rasagiline administered is 0.1-2.5 mg/day. In another embodiment, the amount of rasagiline administered is 0.25-2.0 mg/day. In another embodiment, the amount of rasagiline administered is 0.5-2.0 mg/day. In another embodiment, the amount of rasagiline administered is 0.25 mg/day. In another embodiment, the amount of rasagiline administered is 0.5 mg/day. In another embodiment, the amount of rasagiline administered is 1.0 mg/day. In another embodiment, the amount of rasagiline administered is 1.5 mg/day. In another embodiment, the amount of rasagiline administered is 2.0 mg/day.

In one embodiment, the pridopidine is administered via oral administration.

In one embodiment, the pridopidine is administered through a nasal, inhalation, subcutaneous, intravenous, intraperitoneal, intramuscular, intranasal, buccal, vaginal, rectal, intraocular, intrathecal, topical or intradermal route.

In one embodiment, the pridopidine is administered daily. In another embodiment, the pridopidine is administered more often than once daily. In another embodiment, the pridopidine is administered less often than once daily.

In one embodiment, the amount of pridopidine administered is 0.1-1000 mg/day. In another embodiment, the amount of pridopidine administered is greater than 135 mg/day. In another embodiment, the amount of pridopidine administered is 180-225 mg/day. In another embodiment, the amount of pridopidine administered is 20-180 mg/day. In another embodiment, the amount of pridopidine administered is 30-120 mg/day. In another embodiment, the amount of pridopidine administered is 45-90 mg/day. In another embodiment, the amount of pridopidine administered is 0.1-70 mg/day. In another embodiment, the amount of pridopidine administered is 10-80 mg/day. In another embodiment, the amount of pridopidine administered is 1, 5, 15, 20, 30, 50, 100, or 300 mg.

In one embodiment, the amount of pridopidine administered is about 45 mg/day. In another embodiment, the amount of pridopidine administered is 45 mg/day. In another embodiment, the amount of pridopidine administered less than 45 mg/day.

In one embodiment, the amount of pridopidine administered is about 90 mg/day. In another embodiment, the amount of pridopidine administered is 90 mg/day. In another embodiment, the amount of pridopidine administered is less than 90 mg/day.

In one embodiment, administration of pridopidine is effected twice a day at half the amount.

In one embodiment, administration of pridopidine is effected once every 5 to 9 days.

In one embodiment, a loading dose of an amount different form the intended dose is administered for a period of time at the start of the periodic administration. In another embodiment, the loading dose is double the amount of the intended dose. In another embodiment, the loading dose is half the amount of the intended dose.

In one embodiment, further comprising administration of antidepressant, a psychotropic drug, an antipsychotic, amisulpride, haloperidol, olanzapine, risperidone, sulpiride, or tiapride.

In one embodiment, the periodic administration of rasagiline and pridopidine continues for at least 3 days. In another embodiment, the periodic administration of rasagiline and pridopidine continues for more than 30 days. In another embodiment, the periodic administration of rasagiline and pridopidine continues for more than 42 days. In another embodiment, the periodic administration of rasagiline and pridopidine continues for 8 weeks or more. In another embodiment, the periodic administration of rasagiline and pridopidine continues for at least 12 weeks. In another embodiment, the periodic administration of rasagiline and pridopidine continues for at least 24 weeks. In another embodiment, the periodic administration of rasagiline and pridopidine continues for more than 24 weeks. In another embodiment, the periodic administration of rasagiline and pridopidine continues for 6 months or more.

This invention provides a package comprising a) a first pharmaceutical composition comprising an amount of rasagiline and a pharmaceutically acceptable carrier; b) a second pharmaceutical composition comprising an amount of pridopidine and a pharmaceutically acceptable carrier; and c) instructions for use of the first and second pharmaceutical compositions together to treat a human patient afflicted with a neurodegenerative disorder.

In one embodiment, the neurodegenerative disorder is Huntington's disease.

In one embodiment, the first pharmaceutical composition, the second pharmaceutical composition, or both the first and second pharmaceutical composition is in the form of an aerosol or inhalable powder.

In one embodiment, the first pharmaceutical composition, the second pharmaceutical composition, or both the first and second pharmaceutical composition is in liquid form.

In one embodiment, the first pharmaceutical composition, the second pharmaceutical composition, or both the first and second pharmaceutical composition is in solid form.

In one embodiment, the first pharmaceutical composition, the second pharmaceutical composition, or both the first and second pharmaceutical composition is in capsule form.

In one embodiment, the first pharmaceutical composition, the second pharmaceutical composition, or both the first and second pharmaceutical composition is in tablet form.

In one embodiment, the first pharmaceutical composition further comprises mannitol.

In one embodiment, the first pharmaceutical composition further comprises a filler.

In one embodiment, the amount of rasagiline in the first composition is less than 0.5 mg. In another embodiment, the amount of rasagiline in the composition is 0.01-20.0 mg. In another embodiment, the amount of rasagiline in the first composition is 0.1-2.5 mg. In another embodiment, the amount of rasagiline in the first composition is 0.25-2.0 mg. In another embodiment, the amount of rasagiline in the first composition is 0.5-2.0 mg. In another embodiment, the amount of rasagiline in the first composition is 0.25 mg. In another embodiment, the amount of rasagiline in the first composition is 0.5 mg. In another embodiment, the amount of rasagiline in the first composition is 1.0 mg. In another embodiment, the amount of rasagiline in the first composition is 1.5 mg. In another embodiment, the amount of rasagiline in the first composition is 2.0 mg.

In one embodiment, the amount of pridopidine in the second composition is 0.1-1000 mg. In another embodiment, the amount of pridopidine in the second composition is 20-180 mg. In another embodiment, the amount of pridopidine in the second composition is 30-120 mg. In another embodiment, the amount of pridopidine in the second composition is 45-90 mg. In another embodiment, the amount of pridopidine in the second composition is 0.1-70 mg. In another embodiment, the amount of pridopidine in the second composition is 10-80 mg.

In one embodiment, the amount of pridopidine in the second composition is about 45 mg. In another embodiment, the amount of pridopidine in the second composition is 45 mg. In another embodiment, the amount of pridopidine in the second composition is less than 45 mg.

In one embodiment, the amount of pridopidine in the second composition is about 90 mg. In another embodiment, the amount of pridopidine in the second composition is 90 mg. In another embodiment, the amount of pridopidine in the second composition is less than 90 mg.

In one embodiment, the amount of pridopidine in the second composition is 1, 5, 15, 20, 30, 50, 100, or 300 mg.

The invention also provides rasagiline for use as an add-on therapy or in combination with pridopidine in treating a human patient afflicted with Huntington's disease.

The invention also provides a pharmaceutical composition comprising an amount of rasagiline and an amount of pridopidine for use in treating a human patient afflicted with a neurodegenerative disease, wherein the rasagiline and the pridopidine are administered simultaneously or contemporaneously.

In one embodiment, the neurodegenerative disorder is Huntington's disease.

The invention also provides a pharmaceutical composition comprising an amount of rasagiline and an amount of pridopidine.

In one embodiment, the pharmaceutical composition is in liquid form. In another embodiment, the pharmaceutical composition is in solid form. In another embodiment, the pharmaceutical composition is in capsule form. In another embodiment, the pharmaceutical composition is in tablet form.

In one embodiment, the pharmaceutical composition further comprises mannitol.

In one embodiment, the amount of rasagiline in the composition is less than 0.5 mg. In another embodiment, the amount of rasagiline in the composition is 0.01-20.0 mg. In another embodiment, the amount of rasagiline in the composition is 0.1-2.5 mg. In another embodiment, the amount of rasagiline in the composition is 0.25-2.0 mg. In another embodiment, the amount of rasagiline in the composition is 0.5-2.0 mg. In another embodiment, the amount of rasagiline in the composition is 0.25 mg. In another embodiment, the amount of rasagiline in the composition is 0.5 mg. In another embodiment, the amount of rasagiline in the composition is 1.0 mg. In another embodiment, the amount of rasagiline in the composition is 1.5 mg. In another embodiment, the amount of rasagiline in the composition is 2.0 mg.

In one embodiment, the amount of pridopidine in the composition is 0.1-1000 mg. In another embodiment, the amount pridopidine in the composition is 20-180 mg. In another embodiment, the amount of pridopidine in the composition is 30-120 mg. In another embodiment, the amount of pridopidine in the composition is 45-90 mg. In another embodiment, the amount of pridopidine in the composition is 0.1-70 mg. In another embodiment, the amount of pridopidine in the composition is 10-80 mg. In one embodiment, the amount of pridopidine in the composition is 1, 5, 15, 20, 30, 50, 100, or 300 mg.

In one embodiment, the amount of pridopidine in the composition is about 45 mg. In another embodiment, the amount of pridopidine in the composition is 45 mg. In another embodiment, the amount pridopidine in the composition is less than 45 mg.

In one embodiment, the amount of pridopidine in the composition is about 90 mg. In another embodiment, the amount of pridopidine in the composition is 90 mg. In another embodiment, the amount of pridopidine in the composition is less than 90 mg.

The invention also provides a use of an amount of rasagiline and an amount of pridopidine in the preparation of a combination for treating a human patient afflicted with a neurodegenerative disorder wherein the rasagiline or pharmaceutically acceptable salt thereof and the pridopidine are administered simultaneously or contemporaneously.

The invention also provides a pharmaceutical composition comprising an amount of rasagiline for use in treating a subject afflicted with a neurodegenerative disorder as an add-on therapy or in combination with pridopidine by periodically administering the pharmaceutical composition and the pridopidine to the subject.

The invention also provides a pharmaceutical composition comprising an amount of pridopidine for use treating a subject afflicted with a neurodegenerative disorder as an add-on therapy or in combination with rasagiline by periodically administering the pharmaceutical composition and the rasagiline to the subject.

This invention also provides a therapeutic package for dispensing to, or for use in dispensing to, a subject afflicted with a neurodegenerative disorder or presenting a clinically isolated syndrome, which comprises: a) one or more unit doses, each such unit dose comprising: i) an amount of rasagiline and ii) an amount of pridopidine, wherein the respective amounts of said rasagiline and said pridopidine in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

In the methods, pharmaceutical compositions, packages, and uses as described herein, the rasagiline can be partly or fully deuterium-enriched. In an embodiment, rasagiline has deuterium enrichment of no less than about 10%. In another embodiment, rasagiline has deuterium enrichment of no less than about 50%. In another embodiment, rasagiline has deuterium enrichment of no less than about 90%. In another embodiment, rasagiline has deuterium enrichment of no less than about 98%. A Deuterium-enriched form of rasagiline is described in U.S. Application Publication 2012-0101168 which is hereby incorporated by reference in its entirety into this application.

In the methods, pharmaceutical compositions, packages, and uses as described herein, the pridopidine can be partly or fully deuterium-enriched. In an embodiment, pridopidine has deuterium enrichment of no less than about 10%. In another embodiment, pridopidine has deuterium enrichment of no less than about 50%. In another embodiment, pridopidine has deuterium enrichment of no less than about 90%. In another embodiment, pridopidine has deuterium enrichment of no less than about 98%. Deuterium-enriched forms of pridopidine are described in e.g., PCT International Application Publication Nos. WO 2012/028635 and WO 2011/107583, which are hereby incorporated by reference in their entireties into this application.

For the foregoing embodiments, each embodiment disclosed herein is contemplated as being applicable to each of the other disclosed embodiments. In addition, the elements recited in the packaging and pharmaceutical composition embodiments can be used in the method and use embodiments described herein.

### Pridopidine

Pridopidine mixtures, compositions, the process for the manufacture thereof, the use thereof for treatment of various conditions, and the corresponding dosages and regimens are described in, e.g., PCT International Application Publication Nos. WO 2001/46145, WO 2011/107583, WO 2006/040155, U.S. Patent Application Publication 2011/0206782, U.S. Patent Application Publication 2010/0197712, each of which is hereby incorporated by reference in its entireties into this application.

### Rasagiline

R(+)-N-propargyl-1-aminoindan (“R-PAI”), also known as rasagiline, is a small molecule having the following chemical structure:

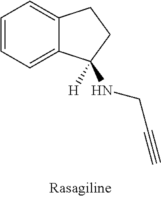

Rasagiline has been reported to be a selective inhibitor of the B-form of the enzyme monoamine oxidase (“MAO-B”) and is useful in treating Parkinson's disease and various other conditions by inhibition of MAO-B in the brain.

A pharmaceutically acceptable salt of rasagiline, rasagiline mesylate, and the process of preparing the same has been described in U.S. Pat. No. 7,855,233, the entire content of which is hereby incorporated by reference.

Crystalline rasagiline, and the process of preparing the same has been described in U.S. Pat. Nos. 7,750,051, 7,968,749, the entire contents of which are hereby incorporated by reference.

Delayed release rasagiline formulations have been described in United States Application Publication Nos. 2009/0181086, 2010/0189790, 2010/0189788, 2010/0189787, and 2010/0189791, the entire content of each of which is hereby incorporated by reference.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds); Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject afflicted with Huntington's disease using rasagiline with pridopidine which provides a more efficacious treatment than each agent alone. The use of rasagiline for Huntington's disease had been previously suggested (Dimpfel, 2011). However, the inventors have surprisingly found that the combination of rasagiline and pridopidine is particularly effective for the treatment of Huntington's disease as compared to each agent alone.

### TERMS

As used herein, and unless stated otherwise, each of the following terms shall have the definition set forth below.

As used herein, “rasagiline” means rasagiline base or a pharmaceutically acceptable salt thereof.

As used herein, a “pharmaceutically acceptable salt” of rasagiline includes citrate, tannate, malate, mesylate, maleate, fumarate, tartrate, esylate, p-toluenesulfonate, benzoate, acetate, phosphate and sulfate salts. For the preparation of pharmaceutically acceptable acid addition salts of the compounds of the invention, the free base can be reacted with the desired acids in the presence of a suitable solvent by conventional methods.

As used herein, an “amount” or “dose” of rasagiline as measured in milligrams refers to the milligrams of rasagiline base present in a preparation, regardless of the form of the preparation. A “dose of 1.0 mg rasagiline” means the amount of rasagiline base in a preparation is 1.0 mg, regardless of the form of the preparation. Thus, when in the form of a salt, e.g. a rasagiline mesylate salt, the weight of the salt form necessary to provide a dose of 1.0 mg rasagiline would be greater than 1.0 mg (e.g., 1.56 mg) due to the presence of the additional salt ion.

As used herein, “about” in the context of a numerical value or range means±10% of the numerical value or range recited or claimed.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the rasagiline and the pridopidine. In this case, the combination may be the admixture or separate containers of the rasagiline and the pridopidine that are combined just prior to administration. Contemporaneous administration refers to the separate administration of the rasagiline and the pridopidine at the same time, or at times sufficiently close together that a synergistic activity relative to the activity of either the rasagiline or the pridopidine alone is observed.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding rasagiline therapy to a patient already receiving pridopidine therapy.

As used herein, “effective” when referring to an amount of rasagiline and/or pridopidine refers to the quantity of rasagiline and/or pridopidine that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition.

“Treating” as used herein encompasses, e.g., inducing inhibition, regression, or stasis of a disease or disorder, e.g., Huntington's disease, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

As used herein, “a subject afflicted with a neurodegenerative disorder” means a subject who has been clinically diagnosed to have a neurodegenerative disorder.

As used herein, a subject at “baseline” is as subject prior to administration of rasagiline.

An HD patient's motor function can be assessed by the Unified Huntington's Disease Rating Scale (UHDRS) Motor Score or “modified motor score (mMS)” derived from the UHDRS Total Motor Score (UHDRS, TMS). UHDRS is a research tool which has been developed by the HSG to provide a uniform assessment of the clinical features and course of HD. The modified motor score is a modified version of the UHDRS made up of 19 items out of the 31 items on the UHDRS motor score. The modified Motor Score is made up of negative motor features such as bradykinesia, rigidity, hand function, eye movements, and gait. The 12 items not included in the mMS but included in the UHDRS total motor score (TMS) include chorea and dystonia, which may differ in their progression from the 19 items on the mMS. The UHDRS is described in, e.g., Huntington Study Group (1996) “Unified Huntington's Disease Rating Scale: Reliability and Consistency” Movement Disorders 11(2):136-142, which is hereby incorporated by reference in its entirety into this application.

A “symptom” associated with a neurodegenerative disorder includes any clinical or laboratory manifestation associated with the neurodegenerative disorder and is not limited to what the subject can feel or observe. For example, a symptom of Huntington's disease includes, but is not limited to, a patient's mMS, motor function as measured by, e.g., the UHDRS-TMS, cognitive function, anxiety or depression. “improvement of” or “improving” a symptom as used herein refers to a favorable change in the patient's symptom as compared to baseline or as compared to a control subject not receiving the treatment.

As used herein, “substantially proceeds administration” as used herein means that the administration of one agent precedes another agent; and the two agents are not administered simultaneously or contemporaneously.

“Polyglutamine disease” as used herein encompasses any inherited disorders characterized by an expanded CAG triplet repeat which codes for a long glutamine repeat including but not limited to Huntington's disease, spinobulbar muscular atrophy (SBMA), and dentatorubral pallidoluysian atrophy. Chai et al. (1999) “Analysis of the Role of Heat Shock Protein (Hsp) Molecular Chaperones in Polyglutamine Disease,” Journal of Neuroscience 19(23):10338-10347, which is hereby incorporated by reference in its entirety into this application.

“Proteinopathy” as used herein encompasses any disease caused by a misfolding and/or aggregation of proteins.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### Animal Models of Huntington's Disease

Most animal models of HD fall into two broad categories, genetic and non-genetic. Historically, nongenetic models have dominated the field of HD research, and typically induce cell death either by excitotoxic mechanisms of by disruption of mitochondrial machinery. Quinolinic acid and kainic acid have been the two most commonly used excitotoxic agents in both rodent and primate models of HD (Ramaswamy, 2007). Emerging molecular technology has enabled the development of genetic murine and, more recently, rat models that attempt to capture the hereditary nature of HD. There are two main categories of genetic mouse models, transgenic and knock-in. Transgenic mice results from the random insertion of a portion of the human htt gene, containing the polyglutamine repeat, in the mouse genome, the expression of which can be driven by different promoters. Alternatively, “knocking in” a portion of the human htt gene in the mouse htt gene locus on chromosome 7 results in the creation of knock-in mice. Transgenic models include transgenic mice model R6/2, R6/1, N171-82Q, YAC, and transgenic rat. Knock-in models include HdhQ92 mouse, HdhQ111 mouse, CAG140 mouse and CAG15O mouse (Ramaswamy, 2007).

### Example 1.1

### Toxin models of HD

A quinolinic acid (QA) rat model is periodically administered an amount of rasagiline and an amount pridopidine. The periodic administration of rasagiline and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the rat than when pridopidine alone or rasagiline alone is administered at the same repetitive dose.

A 3-Nitro-propionic acid (3-NP) rat model is periodically administered an amount of rasagiline and an amount pridopidine. The periodic administration of rasagiline and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the rat than when pridopidine alone or rasagiline alone is administered at the same repetitive dose.

### Example 1.2

### Transgenic Models of HD

A R6/2 mouse model is periodically administered an amount of rasagiline and an amount pridopidine. The periodic administration of rasagiline and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the mouse than when pridopidine alone or rasagiline alone is administered at the same repetitive dose.

### Example 1.3

### Knock-in Mouse Models of HD

A CAG150 mouse model is periodically administered an amount of rasagiline and an amount pridopidine. The periodic administration of rasagiline and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the mouse than when pridopidine alone or rasagiline alone is administered at the same repetitive dose.

### Example 2

### Add-on Therapy for Treating Huntington's Disease

Periodic oral administration of rasagiline (1.0 mg/day) as an add-on therapy for a human patient afflicted with HD who is already receiving pridopidine (45 mg once daily or 45 mg twice a day) provides a clinically meaningful advantage and is more effective (provides at least an additive effect or more than an additive effect) in treating the patient than when pridopidine is administered alone (at the same dose).

Periodic administration of pridopidine (45 mg once daily or 45 mg twice a day) as an add-on therapy for a human patient afflicted with HD who is already receiving rasagiline (1.0 mg/day) provides a clinically meaningful advantage and is more effective (provides at least an additive effect or more than an additive effect) in treating the patient than when rasagiline is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 3

### Add-on Therapy for Treating Huntington's Disease

Periodic oral administration of rasagiline (1.0 mg/day) as an add-on therapy for a human patient afflicted with HD who is already receiving pridopidine (67.5 mg once daily or 67.5 mg twice a day) provides a clinically meaningful advantage and is more effective (provides at least an additive effect or more than an additive effect) in treating the patient than when pridopidine is administered alone (at the same dose).

Periodic administration of pridopidine (67.5 mg once daily or 67.5 mg twice a day) as an add-on therapy for a human patient afflicted with HD who is already receiving rasagiline (1.0 mg/day) provides a clinically meaningful advantage and is more effective (provides at least an additive effect or more than an additive effect) in treating the patient than when rasagiline is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 4

### Add-on Therapy for Treating Huntington's Disease

Periodic oral administration of rasagiline (1.0 mg/day) as an add-on therapy for a human patient afflicted with HD who is already receiving pridopidine (90 mg once daily or 90 mg twice a day) provides a clinically meaningful advantage and is more effective (provides at least an additive effect or more than an additive effect) in treating the patient than when pridopidine is administered alone (at the same dose).

Periodic administration of pridopidine (90 mg once daily or 90 mg twice a day) as an add-on therapy for a human patient afflicted with HD who is already receiving rasagiline (1.0 mg/day) provides a clinically meaningful advantage and is more effective (provides at least an additive effect or more than an additive effect) in treating the patient than when rasagiline is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 5

### Add-on Therapy for Treating Huntington's Disease

Periodic oral administration of rasagiline (1.0 mg/day) as an add-on therapy for a human patient afflicted with HD who is already receiving pridopidine (112.5 mg once daily or 112.5 mg twice a day) provides a clinically meaningful advantage and is more effective (provides at least an additive effect or more than an additive effect) in treating the patient than when pridopidine is administered alone (at the same dose).

Periodic administration of pridopidine (112.5 mg once daily or 112.5 mg twice a day) as an add-on therapy for a human patient afflicted with HD who is already receiving rasagiline (1.0 mg/day) provides a clinically meaningful advantage and is more effective (provides at least an additive effect or more than an additive effect) in treating the patient than when rasagiline is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 6

### Combination Therapy for Treating Huntington's Disease

HD is a fatal neurodegenerative disease characterized by uncoordinated and uncontrollable movements, cognitive deterioration, and behavioral and/or psychological problems. The classic onset of HD symptoms typically occurs in middle age, but the disease also manifests in children and the elderly. Disease progression is characterized by a gradual decline in motor control, cognition, and mental stability and generally results in death within 15-25 years of clinical diagnosis.

HD is a genetic disease, transmitted via autosomal-dominant inheritance. The defective gene, found on chromosome 4, causes the production of a mutant protein, huntingtin (Htt), which aggregates in the central nervous system (CNS) and results in the pathogenesis of HD. The prevalence of HD is approximately 10 per 100,000 in the US and Europe. The only currently marketed product in the United States indicated for HD is tetrabenazine, which has no effect on non-choreic symptoms and disease progression, and is associated with serious side effects such as suicidality and depression. Significant unmet medical needs remain in the development of alternative treatments for HD.

Huntexil® (pridopidine/ACR16) is a drug candidate being developed for the symptomatic treatment of hand movement, balance and gait disturbances in HD. Previous trials in the United States, Europe and Canada demonstrate significant symptomatic relief for patients with HD including improved hand movements and improved gait and balance. These results were observed without any side effects such as sedation and depression seen with other therapies such as neuroleptics and tetrabenzine.

Disclosed herein is the use of rasagiline in addition to or in combination with pridopidine for the treatment of HD.

Periodic oral administration of rasagiline (1.0 mg/day) in combination with pridopidine (45 mg once daily or 45 mg twice a day) to a human patient afflicted with HD provides increased efficacy (provides at least an additive effect or more than an additive effect) in treating the patient than when pridopidine is administered alone or when rasagiline is administered alone (at the same dose).

The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when rasagiline or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 7

### Combination Therapy for Treating Huntington's Disease

Disclosed herein is the use of rasagiline in addition to or in combination with pridopidine for the treatment of HD.

Periodic oral administration of rasagiline (1.0 mg/day) in combination with pridopidine (67.5 mg once daily or 67.5 mg twice a day) to a human patient afflicted with HD provides increased efficacy (provides at least an additive effect or more than an additive effect) in treating the patient than when pridopidine is administered alone or when rasagiline is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when rasagiline or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 8

### Combination Therapy for Treating Huntington's Disease

Disclosed herein is the use of rasagiline in addition to or in combination with pridopidine for the treatment of HD.

Periodic oral administration of rasagiline (1.0 mg/day) in combination with pridopidine (90 mg once daily or 90 mg twice a day) to a human patient afflicted with HD provides increased efficacy (provides at least an additive effect or more than an additive effect) in treating the patient than when pridopidine is administered alone or when rasagiline is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when rasagiline or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 9

### Combination Therapy for Treating Huntington's Disease

Disclosed herein is the use of rasagiline in addition to or in combination with pridopidine for the treatment of HD.

Periodic oral administration of rasagiline (1.0 mg/day) in combination with pridopidine (112.5 mg once daily or 112.5 mg twice a day) to a human patient afflicted with HD provides increased efficacy (provides at least an additive effect or more than an additive effect) in treating the patient than when pridopidine is administered alone or when rasagiline is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when rasagiline or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### REFERENCES

1. ClinicalTrials.gov—Open-label Extension Study of Pridopidine (ACR16) in the Symptomatic Treatment of Huntington Disease (OPEN-HART), retrieved Sep. 26, 2012 <http://www.clinicaltrials.gov/ct2/show/NCT01306929?term=NCT01306929&rank=1>.
2. de Yebenes et al., (2011) “Pridopidine for the treatment of motor function in patients with Huntington's disease (MermaiHD): a phase 3, randomized, double-blind, placebo-controlled trial,” The Lancet Neurology 10(2): 1049-1057.
3. Dimpfel & Hoffmann (2011) “Effects of rasagiline, its metabolite aminoindan and selegiline on glutamate receptor mediated signaling in the rat hippocampus slice in vitro,” BMC Pharmacology 11(2):1-10.
4. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., Nov. 1999<http://www.fda.gov/cber/gdlns/metabol.pdf>.
5. “Huntexil®” The NeurosSearch website, retrieved Sep. 24, 2012, <http://neurosearch.com/Default.aspx?ID=8172>.
6. “Huntington's disease” The NeurosSearch website, retrieved Sep. 24, 2012, <http://neurosearch.com/Default.aspx?ID=8172>.
7. Miller & Bezprozvanny (2010) “Corticostriatal circuit dysfunction in Huntington's disease: intersection of glutamate, dopamine and calcium,” Future Neurol. 5(5): 735-756.
8. NeuroSearch—The HART Study, retrieved Sep. 26, 2012 <http://neurosearch.com/Default.aspx?ID=8490>.
9. PCT International Application Publication No. WO 2007/047863, published Apr. 26, 2007.
10. PCT International Application Publication No. WO 2007/146248, published Dec. 21, 2007, international filing date Jun. 12, 2007.
11. Porter, Robert S. (2011). Huntington's disease. In The Merck manual of diagnosis and therapy (19^{th} ed.) pps 1763-1765.
12. Ramaswamy et al. (20070 “Animal Models of Huntington's Disease” ILAR Journal, 48(4):356-373.
13. Tyagi et al., (2010) “Symptomatic Treatment and Management of Huntington's Disease: An Overview” Global Journal of Pharmacology, 4(1):06-12.
14. PCT International Application Publication WO 2012-028635, published Mar. 8, 2012.
15. U.S. Patent Application Publication No. 2011-0206782, published Aug. 25, 2011 (Zhang).
16. U.S. Patent Application Publication No. 2012-0101168, published Apr. 26, 2012 (Bahar, et al.)
17. U.S. Pat. No. 7,884,208, issued Feb. 8, 2011 (Frenkel et al.).
18. U.S. Pat. No. 7,815,942, issued Oct. 19, 2010 (Peskin et al.)
19. Venuto et al., (2012) “Pharmacologic Approaches to the Treatment of Huntington's Disease,” Movement Disorders 27(1): 31.

1. A method of treating a human patient afflicted with neurodegenerative disorder comprising periodically administering to the patient an amount of rasagiline and an amount of pridopidine, wherein the amounts when taken together are effective to treat the human patient.

2. The method of claim 1, wherein the amount of rasagiline and the amount of pridopidine when taken together is more effective to treat the human patient than when each agent is administered alone.

3. The method of claim 1, wherein each of the amount of rasagiline when taken alone, and the amount of pridopidine when taken alone, is effective to treat the human patient.

4. The method of claim 1, wherein either the amount of rasagiline when taken alone, or the amount of pridopidine when taken alone is not effective to treat a human patient.

5. The method of claim 1, wherein the neurodegenerative disorder is a polyglutamine disease.

6. The method of claim 1, wherein the neurodegenerative disorder is a proteinopathy.

7. The method of claim 1, wherein the neurodegenerative disorder is Parkinson's disease, Alzheimer's disease, Amyotorphic lateral sclerosis (ALS) or Huntington's disease.

8. The method of claim 7, wherein the neurodegenerative disorder is Huntington's disease.

9. The method of claim 1, wherein the amount of rasagiline and the amount of pridopidine when taken together is effective to reduce a symptom of the neurodegenerative disorder in the human patient.

10-15. (canceled)

16. The method of claim 1, wherein the administration of rasagiline substantially precedes the administration of pridopidine.

17. The method of claim 1, wherein the administration of pridopidine substantially precedes the administration of rasagiline.

18. The method of claim 17, wherein the human patient is receiving pridopidine therapy prior to initiating rasagiline therapy.

19. The method of claim 17, wherein the human patient is receiving rasagiline therapy prior to initiating pridopidine therapy.

20. The method of claim 1, wherein the administration of rasagiline and pridopidine improves a symptom of a neurodegenerative disorder by at least 30%-1000%.

21-29. (canceled)

30. The method of any claim 1, wherein the amount of rasagiline administered is 0.01-20.0 mg/day.

31-43. (canceled)

44. The method of claim 1, wherein the amount pridopidine administered is 0.1-1000 mg/day.

45-51. (canceled)

52. The method of claim 1, wherein a loading dose of an amount different form the intended dose is administered for a period of time at the start of the periodic administration.

53-63. (canceled)

64. A package comprising
(a) a first pharmaceutical composition comprising an amount of rasagiline and a pharmaceutically acceptable carrier;
(b) a second pharmaceutical composition comprising an amount of pridopidine and a pharmaceutically acceptable carrier; and
(c) instructions for use of the first and second pharmaceutical compositions together to treat a human patient afflicted with a neurodegenerative disorder.

65-91. (canceled)

92. A pharmaceutical composition comprising an amount of rasagiline and an amount of pridopidine.

93-123. (canceled)
